(19) United States
(12) Patent Application Publication
(10) Pub. No.: US 2026/0248551 A1
(43) Pub. Date: Aug. 27, 2026

(54) DETECTING PROBE ADVANCEMENT USING LEAKAGE CURRENT DETECTION

(71) Applicant: Boston Scientific Scimed, Inc., Maple Grove, MN (US)

(72) Inventor: Jin Yu, Valley Village, CA (US)

(21) Appl. No.: 19/549,983

(22) Filed: Feb. 25, 2026

Related U.S. Application Data
(60) Provisional application No. 63/763,710, filed on Feb. 26, 2025.

(51) Int. Cl. A61B 18/14; A61B 18/00

(52) U.S. Cl. CPC A61B 18/14; A61B 2018/00577; A61B 2018/00642; A61B 2018/00702; A61B 2018/00875

(57) ABSTRACT

Systems and methods for detecting probe position in radiofrequency (RF) ablation systems through leakage current detection. The systems include an RF generator having multiple connector ports with electrical pins that connect to RF probes, each probe having electrodes connected to corresponding pins. The system detects probe position by measuring leakage current between electrical pins, calculating impedance based on the detected current, and comparing measured values to predetermined ranges indicating probe location. The system applies different voltage levels to verify position through changes in measured impedance. Based on detected position, the system can automatically enable or prevent RF power delivery, enhancing procedural safety.

### CROSS REFERENCE TO RELATED APPLICATIONS

This application claims priority to U.S. Provisional Patent Application No. 63/763,710 entitled “DETECTING PROBE ADVANCEMENT USING LEAKAGE CURRENT DETECTION,” filed Feb. 26, 2025, which is hereby incorporated by reference in its entirety.

### TECHNICAL FIELD

The present invention relates generally to tissue ablation systems and methods of operating tissue ablation systems. More specifically, the present invention relates to methods and systems for detecting probe position and advancement in radiofrequency tissue ablation systems using leakage current measurements.

### BACKGROUND

Radiofrequency energy has been used to ablate tissue at various locations within the body. For example, radiofrequency probes have been used to ablate tissue within vertebral bodies to treat chronic back pain, to ablate nerves to treat facet joint pain, or to ablate tissue within joints to treat joint pain within the knees, shoulders, elbows, hips, or ankles.

During radiofrequency ablation procedures, probes must be carefully advanced into the target tissue location before energy delivery can begin. Traditional systems require manual verification of probe placement through imaging or other means to confirm proper positioning. Current automated detection methods typically require closing electrical circuits to measure tissue impedance, adding complexity and potential failure points to the system.

Therefore, there is a need to develop methods and systems that can automatically detect probe position and advancement into target tissue using existing system components, without requiring additional measurement circuits or manual verification steps. Such detection capabilities would enhance both safety and procedural efficiency by providing real-time feedback on probe position during advancement into target tissue.

### SUMMARY

The present invention addresses the need to automatically detect radiofrequency probe position and advancement in tissue ablation systems by utilizing leakage current measurements through switching circuitry. The system's detection capabilities enhance safety and efficiency by providing real-time feedback on probe location without requiring additional measurement components or manual verification steps.

In one aspect, the system of the present invention provides probe detection through leakage current measurement and analysis. The system includes a radiofrequency generator comprising a power source and a controller operatively coupled to the power source and having a processor and a memory element. The radiofrequency generator has switching circuitry that can be placed in an open state while still allowing small leakage currents to flow. At least one radiofrequency probe can be electrically coupled to the radiofrequency generator and has electrodes connected to the switching circuitry. The power source can deliver radiofrequency power to the electrodes sufficient to provide controlled heating of tissue during surgical procedures. The processor executes program instructions that cause the controller to measure impedance between electrodes based on leakage current detected through the open switching circuitry by opening the solid-state relay, based on these measurements, automatically determine whether the probe is positioned within target tissue.

The system implements probe detection strategies including applying different voltage levels to measure impedance through leakage current, comparing measured impedance values against known ranges indicating probe position, utilizing series-connected mechanical relays and switches to enable systematic impedance testing, and distinguishing between in-tissue and out-of-tissue probe positions. These capabilities enable the system to automatically determine probe location and control therapy delivery accordingly, enhancing both safety and usability.

This automated approach eliminates the need for manual position verification while ensuring proper probe placement before enabling radiofrequency power delivery. The system can detect when the probe is properly positioned within target tissue versus outside tissue by comparing measured impedance values to predetermined ranges, automatically adapting its operation accordingly.

In Example 1, a tissue ablation system is provided. The tissue ablation system comprises a radiofrequency generator having a power source and a controller operatively coupled to the power source and having a processor and a memory element. The radiofrequency generator has a plurality of connector ports, wherein each of the plurality of connector ports has a plurality of electrical pins. The tissue ablation system further comprises a radiofrequency probe electrically coupled to a first one of the plurality of connector ports and having first and second electrodes connected to corresponding ones of the plurality of electrical pins. The processor of the tissue ablation system is configured to execute program instructions to detect a leakage current between different electrical pins of the plurality of connector ports; measure an impedance value between the electrodes based on the detected leakage current; compare the measured impedance value to one or more predetermined impedance ranges; and determine whether the radiofrequency probe is positioned within body tissue based on the comparison.

In Example 2, the tissue ablation system of Example 1, wherein the processor is further configured to apply a first voltage level between the electrical pins; measure a first impedance value; apply a second voltage level between the electrical pins; measure a second impedance value; and determine the probe position based on changes between the first and second impedance values.

In Example 3, the tissue ablation system of Example 2, wherein the second voltage level is higher than the first voltage level.

In Example 4, the tissue ablation system of any of Examples 1-3, wherein the predetermined impedance ranges comprise a first range between approximately 2000 ohms and 3500 ohms indicating the probe is positioned within body tissue; and a second range above approximately 16000 ohms indicating the probe is positioned outside body tissue.

In Example 5, the tissue ablation system of any of Examples 1-4, wherein the processor is configured to periodically detect leakage current between the electrical pins to monitor probe position during a procedure.

In Example 6, the tissue ablation system of any of Examples 1-5, wherein the processor is configured to detect when measured impedance values indicate the probe has been removed from body tissue; and prevent activation of radiofrequency power delivery when the probe is detected outside body tissue.

In Example 7, the tissue ablation system of any of Examples 1-6, further comprising a second radiofrequency probe electrically coupled to a second one of the plurality of connector ports and having third and fourth electrodes connected to corresponding ones of the plurality of electrical pins.

In Example 8, the tissue ablation system of Example 7, wherein the processor is configured to detect leakage current between electrical pins connected to the third and fourth electrodes; measure impedance values based on the detected leakage current; and determine position of the second radiofrequency probe based on the measured impedance values.

In Example 9, the tissue ablation system of any of Examples 1-8, wherein measuring the impedance value comprises detecting drop across the pins connected to the first and second electrodes; and calculating the impedance based on the detected voltage drop and the detected leakage current.

In Example 10, the tissue ablation system of any of Examples 1-9, wherein the processor is configured to measure impedance values between approximately 110 ohms and 4200 ohms when the probe is positioned within body tissue.

In Example 11, the tissue ablation system of Example 2, wherein the first voltage level is approximately 2.5V and the second voltage level is approximately 20V.

In Example 12, the tissue ablation system of any of Examples 1-11, wherein the power source is configured to deliver radiofrequency power to the electrodes sufficient to provide controlled heating of tissue during a surgical procedure.

In Example 13, the tissue ablation system of any of Examples 1-12, wherein the processor is configured to store the measured impedance values in the memory element.

In Example 14, the tissue ablation system of any of Examples 1-13, wherein the processor is configured to verify probe position through leakage current measurement during initial probe placement; before enabling radiofrequency power delivery; and upon detection of an impedance change during therapy delivery.

In Example 15, the tissue ablation system of any of Examples 1-14, wherein the processor is configured to periodically interrupt radiofrequency power delivery; perform leakage current measurement during the interruption; and resume radiofrequency power delivery if the measured impedance indicates the probe remains within body tissue.

In Example 16, a tissue ablation system is provided. The tissue ablation system comprises a radiofrequency generator having a power source and a controller operatively coupled to the power source and having a processor and a memory element. The radiofrequency generator has a plurality of connector ports, wherein each of the plurality of connector ports has a plurality of electrical pins. The tissue ablation system further comprises a radiofrequency probe electrically coupled to a first one of the plurality of connector ports and having first and second electrodes connected to corresponding ones of the plurality of electrical pins, wherein the power source is configured to deliver radiofrequency power to the electrodes sufficient to provide controlled heating of tissue during a surgical procedure. The processor of the tissue ablation system is configured to execute program instructions to detect a leakage current between different electrical pins of the plurality of connector ports; measure an impedance value between the electrodes based on the detected leakage current; compare the measured impedance value to one or more predetermined impedance ranges; and determine whether the radiofrequency probe is positioned within body tissue based on the comparison.

In Example 17, the tissue ablation system of Example 16, wherein the processor is further configured to apply a first voltage level between the electrical pins; measure a first impedance value; apply a second voltage level between the electrical pins; measure a second impedance value; and determine the probe position based on changes between the first and second impedance values.

In Example 18, the tissue ablation system of Example 17, wherein the second voltage level is higher than the first voltage level.

In Example 19, the tissue ablation system of Example 16, wherein the predetermined impedance ranges comprise a first range between approximately 2000 ohms and 3500 ohms indicating the probe is positioned within body tissue; and a second range above approximately 16000 ohms indicating the probe is positioned outside body tissue.

In Example 20, the tissue ablation system of Example 16, wherein the processor is configured to periodically detect leakage current between the electrical pins to monitor probe position during a procedure.

In Example 21, the tissue ablation system of Example 16, wherein the processor is configured to detect when measured impedance values indicate the probe has been removed from body tissue; and prevent activation of radiofrequency power delivery when the probe is detected outside body tissue.

In Example 22, the tissue ablation system of Example 16, further comprising a second radiofrequency probe electrically coupled to a second one of the plurality of connector ports and having third and fourth electrodes connected to corresponding ones of the plurality of electrical pins.

In Example 23, the tissue ablation system of Example 22, wherein the processor is configured to detect leakage current between electrical pins connected to the third and fourth electrodes; measure impedance values based on the detected leakage current; and determine position of the second radiofrequency probe based on the measured impedance values.

In Example 24, the tissue ablation system of Example 16, wherein measuring the impedance value comprises detecting drop across the pins connected to the first and second electrodes; and calculating the impedance based on the detected voltage drop and the detected leakage current.

In Example 25, the tissue ablation system of Example 16, wherein the processor is configured to measure impedance values between approximately 110 ohms and 4200 ohms when the probe is positioned within body tissue.

In Example 26, the tissue ablation system of Example 17, wherein the first voltage level is approximately 2.5V and the second voltage level is approximately 20V.

In Example 27, a tissue ablation system is provided. The tissue ablation system comprises a radiofrequency generator comprising a power source and a controller operatively coupled to the power source and having a processor and a memory element, the radiofrequency generator having a plurality of connector ports, wherein each of the plurality of connector ports has a plurality of electrical pins. The tissue ablation system further comprises a first radiofrequency probe electrically coupled to a first one of the plurality of connector ports to form a first channel and having first and second electrodes connected to corresponding ones of the plurality of electrical pins, and a second radiofrequency probe electrically coupled to a second one of the plurality of connector ports to form a second channel and having third and fourth electrodes connected to corresponding ones of the plurality of electrical pins. The power source of the tissue ablation system is configured to deliver radiofrequency power to the electrodes via the corresponding electrical pins. The processor of the tissue ablation system is configured to execute program instructions to detect leakage current between electrical pins of the plurality of connector ports; measure impedance values based on the detected leakage current; determine positions of the first and second radiofrequency probes based on the measured impedance values; and control delivery of radiofrequency power based on the determined positions.

In Example 28, the tissue ablation system of Example 27, wherein the controller is configured to apply a first voltage level of approximately 2.5V across the pins of each channel; measure first impedance values; apply a second voltage level of approximately 20V across the pins of each channel; measure second impedance values; and determine probe positions based on changes between the first and second impedance values.

In Example 29, the tissue ablation system of Example 27, wherein the controller is further configured to the controller is configured to compare measured impedance values to stored impedance ranges corresponding to in-body and out-of-body probe positions.

In Example 30, the tissue ablation system of Example 29, wherein the in-body impedance range is between approximately 2000 ohms and 3500 ohms; and the out-of-body impedance range is above approximately 16000 ohms.

In Example 31, the tissue ablation system of Example 27, wherein the controller is configured to periodically detect leakage current between electrical pins to monitor probe positions during a procedure.

In Example 32, a computer-implemented method of detecting probe position in a tissue ablation system is provided. The tissue ablation system has a radiofrequency generator with a plurality of connector ports, wherein each connector port has a plurality of electrical pins, and a radiofrequency probe having first and second electrodes connected to corresponding pins of a first connector port. The method comprises executing, by a processor of a controller operatively coupled to the radiofrequency generator, program instructions stored in a memory element to: detect leakage current between the pins connected to the first and second electrodes; measure impedance values based on the detected leakage current; compare the measured impedance values to predetermined threshold values; determine whether the probe is positioned within body tissue based on the comparison; and control delivery of radiofrequency energy based on the determined probe position.

In Example 33, the computer-implemented method of Example 32, wherein executing the program instructions further comprises: applying different voltage levels across the pins while detecting leakage current; measuring impedance values at each voltage level; and determining probe position based on changes in measured impedance across the different voltage levels.

In Example 34, the computer-implemented method of Example 32, wherein executing the program instructions further comprises: comparing measured impedance values to: a first range between approximately 2000 ohms and 3500 ohms indicating in-body probe position; and a second range above approximately 16000 ohms indicating out-of-body probe position.

In Example 35, the computer-implemented method of Example 32, wherein executing the program instructions further comprises: continuously monitoring impedance through leakage current detection during a procedure; detecting when measured impedance indicates probe removal from body tissue; and preventing activation of radiofrequency power delivery when the probe is detected outside body tissue.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a schematic block diagram of an exemplary multichannel radiofrequency tissue ablation system, in accordance with the teachings of the present invention;

FIG. 2 is a schematic electrical diagram illustrating an exemplary use of controlling switching elements for probe position detection, in accordance with the teachings of the present invention; and

FIG. 3 is a schematic electrical diagram illustrating equivalent circuit impedances during leakage current measurement for probe position detection, in accordance with the teachings of the present invention.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

A radiofrequency ablation (RFA) procedure is a minimally invasive medical procedure that is used to ablate targeted tissue at a surgical site, and typically involves using one or more energy delivery devices (e.g., radiofrequency (RF) probes) having one or more electrodes associated therewith. The radiofrequency probe can be coupled to one or more radiofrequency (RF) generators to deliver power in the form of radiofrequency energy to the targeted tissue via the electrodes. The radiofrequency energy generates localized heat which is used to target and destroy (e.g., ablate) the targeted tissue. In pain management, the targeted tissue can be nerve tissue responsible for sending pain signals.

The RFA procedure can be utilized to ablate different types of tissue, such as nerve tissue, adipose tissue, muscle tissue, and the like. The RFA procedure can be performed at various locations on an external surface of a subject or patient or within the subject. The RFA procedure performed within the subject can be performed in a minimally invasive manner (e.g., via a percutaneous, laparoscopic, endoscopic, or intravascular approach) or in a more invasive manner (e.g., via an open surgical procedure). For example, Applicant's existing technology (the Intracept® procedure by Relievant® Medsystems, Inc.) offers a safe and effective minimally invasive procedure that targets the basivertebral nerve (BVN) for the relief of chronic low back pain that originates at least partly from one or more endplates of one or more vertebral bodies. The Intracept® procedure involves application of radiofrequency energy from a radiofrequency generator using a bipolar radiofrequency probe, where the applied radiofrequency energy is sufficient to ablate the basivertebral nerve within the vertebral body. The procedure can be performed in multiple different vertebral bodies sequentially or simultaneously using a single radiofrequency probe or using multiple radiofrequency probes coupled to a single generator. The basivertebral nerve trunk can then be ablated by the RF probe. Other intraosseous nerves within the vertebral body that innervate the endplates and/or intervertebral disc can also be targeted and ablated.

The RFA procedure is typically performed using a surgical introducing or access assembly that can include a hollow cannula that forms a sheath and a stylet that is disposed within the cannula until a distal tip of the stylet extends beyond the cannula. The stylet adds rigidity and support to the cannula when inserted into the patient. The stylet helps the surgeon guide the assembly to the surgical site. Once the cannula is in position at the surgical site, then the stylet is removed from the cannula and the RF probe is inserted therein. The RF probe can then deliver RF energy to the targeted tissue at the surgical site. In a bipolar RF probe arrangement, the probe can include a pair of electrodes, including a first active energy delivering electrode and a second return electrode. According to another arrangement, the RF probes can be configured in a monopolar arrangement, where an RF probe employs an active energy delivering electrode and a separate device or element can be used as a return. The return can be configured as a return pad coupled to the subject or a return electrode disposed on a separate RF probe. For example, a first RF probe can be used that includes the active energy delivering electrode and a second RF probe can be used that includes a return electrode. The RF probe can also include additional structure, including for example temperature sensors (e.g., thermocouples or thermistors), that can be employed to sense or detect the temperature at the surgical site during the surgical procedure. The sensed or detected temperature can provide important information to the surgeon about the ablation procedure. The RFA procedure contemplates positioning the two electrodes of the one or more radiofrequency probes within the vertebral body. The RF generator supplies power to the active electrode of the RF probe at a selected power or voltage level and for a time sufficient to create a desired lesion within the vertebral body sufficient to ablate the basivertebral nerve within the vertebral body. In some implementations, the step of applying power to the RF probe includes supplying power for a selected duration or period of time (e.g., 1 minute to 1 hour or for any time within this range) to achieve a selected temperature at the surgical site. The temperature of the thermal energy supplied by the RF probe to the targeted tissue can range from between about 50° C. and about 115° C. (e.g., from about 70° C. to about 90° C., from about 75° C. to about 90° C., from about 83° C. to about 87° C., from about 80° C. to about 100° C., from about 85° C. to about 95° C., from about 90° C. to about 110° C., from about 95° C. to about 115° C., or overlapping ranges thereof). The temperature ramp-up during the surgical procedure can range from between about 0.1-5 degrees Celsius/second (e.g., 0.1-1.0 degrees Celsius/second, 0.25 to 2.5 degrees Celsius/second, 0.5-2.0 degrees Celsius/second, 1.0-3.0 degrees Celsius/second, 1.5-4.0 degree Celsius/second, 2.0-5.0 degrees Celsius/second). The time of treatment can range from between about 10 seconds and about 1 hour (e.g., from 10 seconds to 1 minute, 1 minute to 5 minutes, from 5 minutes to 10 minutes, from 5 minutes to 20 minutes, from 8 minutes to 15 minutes, from 10 minutes to 20 minutes, from 15 minutes to 30 minutes, from 20 minutes to 40 minutes, from 30 minutes to 1 hour, from 45 minutes to 1 hour, or overlapping ranges thereof). Pulsed energy can be delivered as an alternative to or in sequence with continuous energy. For radiofrequency energy, the energy applied by the RF generator can range from between about 350 KHz and about 650 kHz (e.g., from 400 kHz to 600 kHz, from 350 kHz to 500 kHz, from 450 kHz to 550 kHz, from 500 kHz to 650 kHz, overlapping ranges thereof, or any value within the recited ranges, such as 450 KHz±5 kHz, 475 KHz±5 kHz, 487 kHz±5 kHz). The power of the radiofrequency energy generated by the RF generator can range from between about 5 W and about 30 W (e.g., from 5 W to 15 W, from 5 W to 20 W, from 8 W to 12 W, from 10 W to 25 W, from 15 W to 25 W, from 20 W to 30 W, from 8 W to 24 W, and overlapping ranges thereof, or any value within the recited ranges).

Although described primarily in connection with procedures for the treatment within the spine, the systems and methods of the present invention can also be used for radiofrequency tissue ablation procedures intended to treat ailments or conditions other than those associated with the spine or back pain. The procedures can involve ablation of nerves outside of bones but related to the spine (e.g., sacroiliac joints, facet joints, etc.). Target treatment locations within bones other than vertebral bodies may also be accessed. For example, target treatment locations within a humerus, radius, femur, tibia, calcaneus, tarsal bones, hips, knees, phalanges, and/or other orthopedic targets can be accessed. The ablation procedures can include, for example, ablation of nerves within or surrounding other bones other than the vertebral column, cardiac tissue ablation for treatment of atrial fibrillation or other abnormal heart rhythms irregularities, tumor ablation at any location within the body (e.g., within bones, lungs, breasts, thyroids, livers, or other organs or tissues), peripheral nerve ablation, pulmonary artery ablation, renal denervation procedures, uterine fibroid ablation, endometrial ablation, and/or the like. The systems and methods described herein may be used in connection with any radiofrequency procedure during which impedance (e.g., tissue impedance or impedance between two electrodes) is monitored.

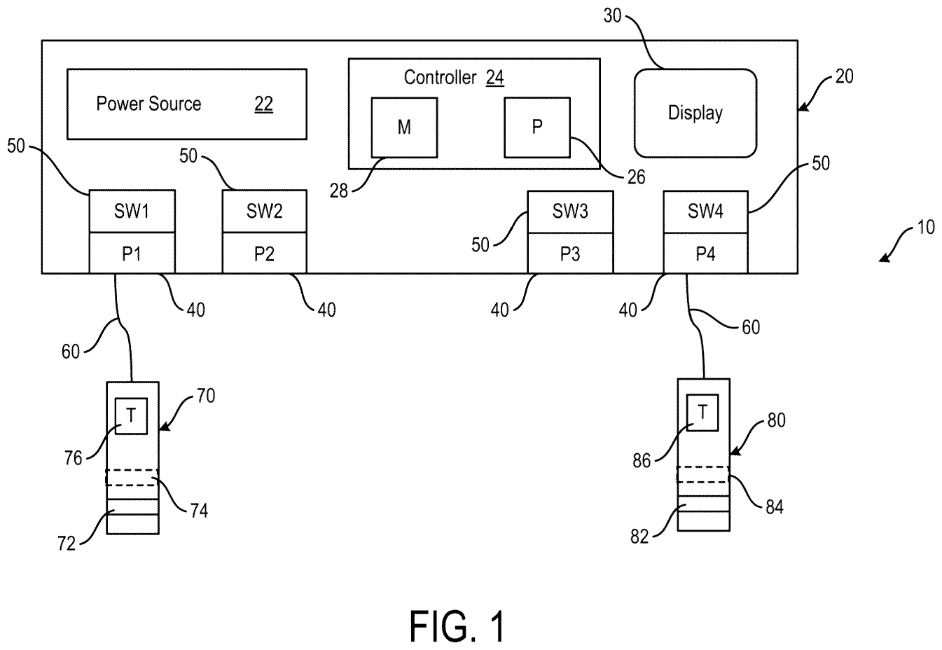

FIG. 1 is a schematic block diagram of a tissue ablation system 10 suitable for use with the present invention. The tissue ablation system includes a generator, such as a radiofrequency generator 20, that is coupled to one or more radiofrequency probes 70, 80. The RF generator 20 can be electrically and communicatively coupled to the RF probes 70, 80 via any suitable conduit 60. The conduits 60 can facilitate bidirectional communication of electrical signals and data or instructions between the RF generator 20 and the RF probes 70, 80. For example, the conduits 60 can include one or more electrical wires or lines (not shown) suitable for conveying the electrical signals. The generator can also be any other type of energy source suitable for supplying ablative energy to the probes. According to one embodiment, the illustrated RF generator 20 can include a power source 22 that can be configured to generate and supply radiofrequency power or energy to the RF probes 70, 80 via the conduits 60 at a desired frequency and power level. The RF generator 20 can also include a controller 24 that includes for example a processor (e.g., CPU) 26 suitable for processing and executing applications and instructions that are stored in a memory element 28. The processor 26 can be any suitable type of processor, such as a special purpose processor, and the memory element 28 can be any suitable type of memory. The processor 26 can be implemented in hardware, software, firmware, or any suitable combination thereof, and can be configured to execute program instructions stored in the memory element 28. The processor 26 and the memory element 28 are shown herein as forming part of the controller 24, but one of ordinary skill in the art will readily recognize that the processor 26 and the memory 28 can form part of other components of the RF generator 20. For the sake of simplicity, only one processor 26 and memory element 28 are shown, although multiple processors and memory elements can also be employed in the RF generator 20. The controller 24 can be configured as described herein, and at least is configured to monitor temperature, power, and/or impedance in connection with a tissue ablation procedure performed by the tissue ablation system 10. The RF generator 20 can further include a user interface or display element 30 suitable for displaying information or instructions to a user (e.g., surgeon) via a series of user interfaces or for allowing the user to provide instructions to the RF generator. As such, the display element 30 can employ a user interface generator 32 for generating the user interfaces based on programmatic instructions stored in the memory element 28 and processed by the processor 28 or by a processor forming part of the display element 30. The display element 30 can be configured to display information to the user. For example, during startup and use, the current status of the RF generator 20 and energy delivery or treatment parameters can be displayed on the display element 30 via one or more user interfaces. During energy delivery, the display element 30 can be configured to display desired treatment time, remaining treatment time, temperature, impedance, and power information (alphanumerically and/or graphically). For example, graphical representations of power vs. time and impedance vs. time can be displayed. The display element 30 can also be separate from the RF generator 20 and need not form part of the generator. Although not shown, the tissue ablation system 10 can also employ additional accessory devices, such as suitable user input devices including a keyboard, mouse, trackpad, voice-activated input device, and the like.

The illustrated RF generator 20 can also have formed therein a plurality or series of connector ports 40 that allow the RF probes 70, 80 to be coupled to the generator. The RF generator 20 can have any selected number of connector ports 40. In the illustrated embodiment, the RF generator 20 includes four probe connector ports 40A and a ground connector port 40B. The connector ports 40 can have any selected size, shape, and configuration. According to one embodiment, the connector ports 40 can employ a series of electrical pins that connect to RF probes and a ground pad and allow electrical energy and data to be exchanged therebetween. For example, a first electrical pin (or multiple electrical pins) can be coupled to the RF probe so as to deliver RF power to an energy delivering electrode when placed thereon and a second electrical pin can communicate with a return electrode when placed thereon. A third electrical pin can be employed to communicate with a temperature sensor in the RF probe and a fourth electrical pin can be used to exchange data between the RF generator and the RF probe. Those of ordinary skill in the art will readily recognize that any suitable type (e.g., Lemo or Din type connectors), arrangement, and number of electrical connectors can be employed in the connector ports 40.

The illustrated RF generator 20 can also employ a switching element module or a series of discrete switching elements 50 that can be used to help control the generation, modulation, and delivery of RF energy. The switching elements 50 help ensure the precise delivery of energy to the target tissue via the RF probes while maintaining system safety and functionality. The switching elements 50 can also control the RF energy delivered to the active energy delivering electrode of the RF probe through an associated connector port 40. For the sake of simplicity, a switching element 50 is shown coupled to and disposed in electrical communication with each illustrated connector port 40. Those of ordinary skill in the art will readily recognize that a separate switching element module that is arranged in electrical communication with each of the connector ports 40 can also be employed. The illustrated switching elements 50 can also be positioned and arranged to manage the activation of circuits connected to sensors in the RF probes 70, 80, such as temperature sensors or impedance measurement systems, and to manage the activation of the ground connector port 40B. The switching elements 50 can also be arranged to route sensor signals to specific monitoring circuits in the controller 24 for processing. The switching elements 50 can also serve to verify proper grounding or return electrode connection before enabling RF output to ensure patient safety. The switching elements 50 can be any suitable type of switching element, such as MOSFETs, IGBTs, Pin diodes, mechanical and solid-state relays, and the like.

The illustrated tissue ablation system 10 can also employ one or more RF probes. For the sake of simplicity and for purposes of explanation, a pair of RF probes 70, 80 are shown connected to the RF generator 20. The RF probes 70, 80 can be the same or can be different depending upon the selected type of surgical procedure being performed and/or the purpose and function of the RF probe. The RF probe 70 has a main body having an outer surface having one or more electrodes mounted thereon. In a bipolar RF probe arrangement, the RF probe 70 can have a first active energy delivering electrode 72 and a return electrode 74 disposed on an outer surface of the main body. The probe can also include a temperature sensor 76 for sensing or measuring temperature at the surgical site. The electrodes and the temperature sensor 76 can be coupled to the RF generator 20 via electrical leads that pass through the conduit 60. If the RF probe 70 is constructed as a monopolar device, then the probe can include a single active electrode 72, which can function as the active energy delivering electrode or as the return electrode. In this type of arrangement, the second electrode 74 (in dashed lines) is either inactivated or not present. Similarly, the RF probe 80 has a main body having an outer surface having one or more electrodes mounted thereon. In a bipolar RF probe arrangement, the RF probe 80 can also have a first active energy delivering electrode 82 and a return electrode 84. The probe can also include a temperature sensor 86. The electrodes and the sensor can also be coupled to the RF generator 20 via electrical leads that pass through the conduit 60. If the RF probe 80 is constructed as a monopolar device, then the probe can also include a single active electrode 82, which can function as the active energy delivering electrode or as the return electrode. In this type of arrangement, the second electrode 84 is either inactivated or not present. Each RF probe that is coupled to a connector port 40 of the RF generator 20 forms in essence a channel. As such, multiple RF probes coupled to multiple connector ports 40 of the RF generator 20 can form multiple channels.

In some embodiments, the RF generator can comprise a four-port RF generator that can connect to probes through multiple pins in each port. The probe can have a tip and ring configuration for its electrodes. The system can use specific pins within each port to connect to these electrodes, for example, Pin 1 can connect to the probe tip and Pin 4 can connect to the probe ring. When switching circuitry in the system is placed in an open state, a small leakage current can flow between Pin 1 and Pin 4. By measuring this leakage current, the system can determine the impedance between the probe's tip and ring electrodes. This enables the system to detect whether the probe is positioned within body tissue through leakage current measurement techniques as describe in the following sections.

The systems and methods described herein are directed to detecting probe position of radiofrequency probes 70 and 80 using leakage current through switching circuitry in radiofrequency tissue ablation systems. The system uses two types of switches that work together on the same circuit path, mechanical relays that physically open and close; and solid-state relays (SSR) that switch electronically. Leakage current can be a small amount of current that still manages to flow even when the SSR is open and mechanical relay is closed. This small flow of current is called SSR leakage current. While traditionally considered an unwanted effect, the leakage current can be used as a tool to detect where probe 70 and 80 is positioned inside the body or outside the body and can serve to control RF power delivery during therapy. When electricity flows through tissue, it encounters resistance, or impedance. Different types of tissues and conditions may create different impedance levels. By measuring the impedance between the probe's electronics using small leakage currents, the system can determine if the probe is surrounded by body tissue or is outside the body.

The controller 24 can be configured to initiate probe position detection at several points during a procedure through control of the switching circuitry. During initial setup, before any RF power delivery is enabled, the system can perform position detection by placing the switching circuitry in an open state to verify proper probe placement through leakage current measurement. The controller 24 maintains this open state configuration long enough to perform voltage level tests and determine probe position based on the measured impedance values.

During therapy delivery, the system can periodically interrupts RF power delivery by opening the switching circuitry to check probe position through leakage current measurement, ensuring the probe remains properly positioned within the target tissue. This periodic checking occurs between RF power delivery cycles without compromising therapy effectiveness.

The switching sequence follows specific safety protocols established and controlled by the controller 24. Before any RF power delivery is initiated, the system verifies proper probe position and grounding status. The switching circuitry's arrangement of mechanical relays and solid-state switches in series provides safety through multiple isolation points. When transitioning between therapy delivery and position detection modes, the controller opens the mechanical relay to establish primary isolation before configuring the solid-state switches for leakage current measurement. This sequential switching approach ensures patient safety while enabling accurate position detection.

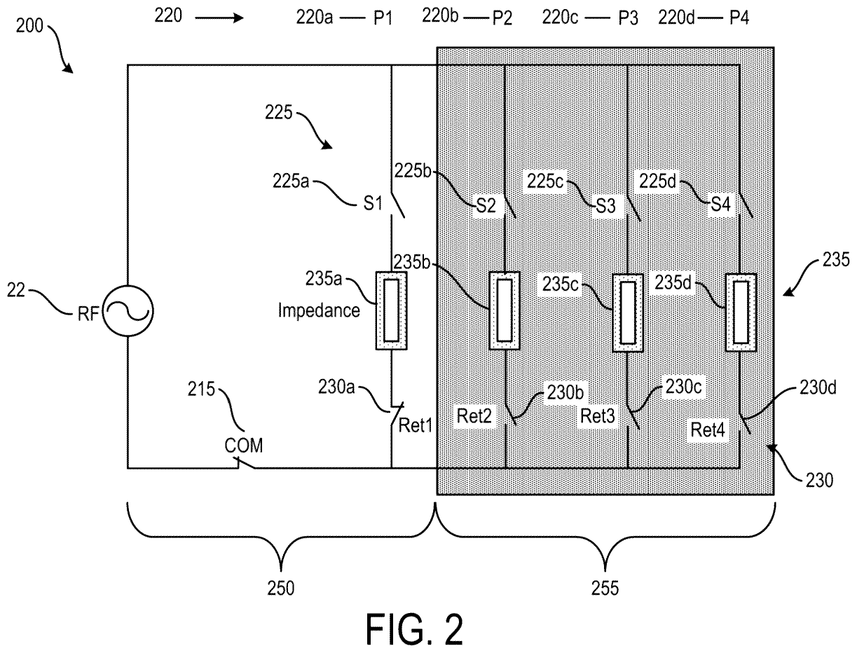

FIG. 2 illustrates an exemplary schematic electrical or circuit diagram 200 of a switching element configuration for probe position detection in the multichannel RF tissue ablation system 10. The illustrated circuit diagram 200 demonstrates an arrangement where a selected number (e.g., pairs) of relays, such as solid-state relays (SSRs), are electrically coupled between an RF power source 22 and the probe electrodes 72, 82. Each port 220a-220d (P1, P2, P3, P4) of the RF generator 20 can include a source SSR 225a-225d (S1, S2, S3, S4) and a corresponding return SSR 230a-230d (Ret1, Ret2, Ret3, Ret4). The RF power source 22 can be coupled to a common circuit point (COM) 215 and optionally an associated relay or switch. At any given period of time, one port 200 of the RF generator 20 can have the associated SSR pair (e.g., source relay and return relay) disposed in a closed position or state to allow current flow, while all other SSR pairs remain disposed in an open position or state. When a port 200 is inactive, the associated source SSR remains or is disposed in an open state. The source SSRs 225 control the connection between the RF power source 22 and their respective ports 220, while the return SSRs 230 complete the circuit path back to the common circuit connection point 215. The paired relay arrangement provides for electrical isolation between ports 220 during both therapy based power delivery and position detection measurements.

Each SSR 225, 230 exhibits characteristics that enable leakage current to flow even when the relays are disposed in an open state. This leakage current creates measurable impedance paths that vary based on whether the probe 220 is positioned within body tissue or outside the body. The source SSRs 225 and return SSRs 230 work together to control the illustrated electrical pathways. When measuring probe position in relation to Port 1 (220a), the source SSR S1 (225a) and return SSR Ret1 (230a) form a complete circuit path, while S2/Ret2, S3/Ret3, and S4/Ret4 maintain their open states. A first voltage can be applied through the source SSR to establish a baseline impedance measurement. The system can then apply a second voltage to obtain additional impedance measurements that help determine probe position. For the circuit configuration shown in FIG. 2, Port 1 (220a) and its associated S1/Ret1 relay pair forms a measurement region 250. Ports P2, P3, P4 (220b-220d) and their associated relay pairs S2/Ret2, S3/Ret3, S4/Ret4 forms an inactive region 255.

Each SSR, when disposed in an open state, presents an equivalent impedance. This characteristic enables measurable leakage current to flow through the circuit even when the SSRs are open, enabling probe position detection without requiring a fully closed circuit. The source SSR 225 for each port 220 connects directly to the RF power source 22, while the return SSR 230 connects to the common circuit point 215. This circuit configuration ensures that impedance measurements reflect the conditions between the probe electrodes rather than other circuit elements.

In the four-port 220 configuration as shown, the system can sequentially test each port while maintaining isolation from the others. The source and return SSR pairs for ports P2, P3, and P4 remain disposed in an open state while measurements are taken through port P1. The illustrated circuit diagram also shows how the switching or relay elements can be arranged to measure impedance through SSR leakage current while also maintaining capability for normal RF power delivery during therapy. This arrangement enables both functions through the same circuit paths. When measuring inactive port impedance, the circuit formed by the open SSR pair (e.g., relays 225 and 230) allows small leakage currents to flow. These leakage currents create voltage drops that can be measured to determine impedance values.

The arrangement of the SSR pairs also provides electrical isolation between ports during both impedance measurement and RF power delivery. Each port's relay elements 225, 230 operate independently, enabling the system to assess probe position at one port without affecting the others. The electrical path for each port 220 includes an impedance element 235a-235d. These impedance elements are resistive components that provide known resistance values for reference measurements. Resistors are commonly used as impedance elements, and can include various types such as precision resistors, wire-wound resistors, or metal film resistors depending on the specific measurement and power requirements of the system. For Port 1 (220a), the impedance element 235a establishes a defined circuit and hence measurement path through which leakage current flows when the SSRs are in an open state. This arrangement allows for consistent impedance measurements across all ports. Each port (P1, P2, P3, P4) has its own impedance element (235a-235d) connected, creating parallel measurement paths that maintain electrical isolation between channels. The impedance elements 235 act as reference points for measurements, provide consistent paths for leakage current flow, and help establish baseline values.

When measuring the current through any port 220, the leakage current from the RF power source 22 flows through both the port's impedance element and any tissue present between the probe electrodes. The total measured impedance reflects the combined effect of the impedance element, the open SSRs, and the tissue condition at the probe location. The impedance elements 235 also contribute to measurement accuracy by providing known reference values. As the leakage current flows through these impedance paths, the system can compare measured values against these references to reliably determine probe position.

Measurements through Port 1 (220a) can be taken while maintaining S2/Ret2, S3/Ret3, and S4/Ret4 in their open states. For measuring at Port 2, S1/Ret1, S3/Ret3, and S4/Ret4 remain open while measurements occur through S2/Ret2. This sequential pattern continues for measurements at Ports 3 and 4.

The RF power source 22 connects through each source SSR to its corresponding port. The return path from each port passes through its associated return SSR to the common point 215. This forms separate measurement paths for each port while maintaining electrical isolation. Between the RF source and COM, each port contains both source and return SSRs connected in series. This series connection of SSRs provides the pathway for both leakage current measurement and RF power delivery when activated. The SSRs maintain a high impedance state when open, yet still allow small leakage currents to flow. These leakage currents provide a means to measure the impedance between probe electrodes without requiring the SSRs to close.

Through the open SSRs, voltage can be applied and resulting currents measured to determine probe position. The measured impedance values differ based on whether the probe electrodes are in contact with body tissue or remain outside the body. When a probe is positioned within body tissue, the measured impedance through the open SSRs falls within characteristic ranges. These ranges differ from those measured when the probe is outside the body due to the electrical properties of tissue. The controller can measure impedance through leakage current detection according to test sequences. Using two different voltage levels enables the system to verify probe position through changes in measured impedance values.

For detecting probe position through leakage current, the system evaluates different measurement conditions, as shown for example in Table 1.

TABLE 1

| | | | | | Open |
|---|---|---|---|---|---|
| | 0 Ohm | 100 Ohm | 1000 Ohm | 1500 Ohm | Circuit |
| Source SSR On, | — | 110 | 865 | 1160 | 1163 |
| Return SSR on | | | | | |
| (Avg. 2.5 V) | | | | | |
| Source SSR On, | 2200 | 2203 | 2543 | 2750 | 3200 |
| Return SSR off | | | | | |
| (Avg. 2.5 V) | | | | | |
| Source SSR On, | 3470 | 3470 | 3664 | 3825 | 4123 |
| Return SSR off | | | | | |
| (Avg. 20 V) | | | | | |
| Source Relay | 16000 | 16600 | 17200 | 18061 | 20374 |
| Off, Return | | | | | |
| relay off (Avg. | | | | | |
| 2.5 V) | | | | | |

The table presents impedance measurements under varying configurations: when both source and return SSRs are on (Source SSR On, Return SSR On) and hence in a closed state, when only source SSR is on (Source SSR On, Return SSR Off), and when all relays are off (Source Relay Off, Return Relay Off) and hence in a closed state.

Table 1 shows impedance values measured across different load conditions ranging from 0 ohm to open circuit. When both source and return SSRs are on with an average voltage of 2.5V, the measured impedance ranges from 110 ohms at 100 ohm load to 1663 ohms at open circuit.

With the source SSR on and return SSR off at 2.5V average voltage, the measured impedance increases to a range of 2200 ohms at 0 ohm load to 3200 ohms at open circuit. When the voltage is increased to 20V average under the same SSR configuration, the impedance values range from 3470 ohms to 4123 ohms.

When both source relay and return relay are off with 2.5V average voltage, the measured impedance values increase, ranging from 16000 ohms to 20374 ohms across the load conditions. These impedance ranges form the basis for probe position detection.

The system can utilize the foregoing impedance measurements to determine probe location. When a probe is within body tissue, the measured impedance through the open switching circuitry falls within the range seen when source SSR is on and return SSR is off-between approximately 2000 ohms and 3500 ohms.

Based on the values shown in Table 1, the controller measures impedance through leakage current detection according to defined test sequences. Using two different voltage levels, 2.5V and 20V, enables the system to verify probe position through changes in measured impedance values. When measuring probe position through leakage current, the system follows the sequence of voltage application and impedance measurement. The process begins with placing the switching circuitry in an open state, where the source SSR remains on while the return SSR is maintained off.

The system first applies a 2.5V signal across the electrodes through the open switching circuitry. At this voltage level, the measured impedance indicates the baseline condition. For a probe positioned within body tissue, this measurement typically falls between 2200 ohms and 3200 ohms as shown in Table 1. Following the initial measurement, the system increases the test voltage to 20V while maintaining the same switching circuit configuration. This higher voltage level produces impedance measurements between 3470 ohms and 4123 ohms when the probe is within body tissue. The change in measured impedance between the two voltage levels provides additional verification of probe position.

These measurements can occur periodically during a procedure to monitor probe position. The system applies the test voltages through the leakage current path created by the open switching circuitry, enabling position detection without interrupting the normal RF power delivery capability.

For an inactive port, the measurement sequence remains the same-applying first 2.5V then 20V through the open switching circuitry. However, when the probe is outside body tissue, the measured impedance values can exceed 16000 ohms at both voltage levels, distinguishing from in-body measurements.

The impedance determination occurs through voltage and current measurements across the open switching circuitry. The system measures the voltage drop resulting from leakage current flow and can determine the impedance based on the switching elements.

In some embodiments, the controller can use these impedance measurements to control RF power delivery. When measured values indicate the probe is within body tissue, the system can enable RF power delivery. If measured impedance exceeds 16000 ohms, indicating the probe is outside body tissue, the system can prevent RF power activation.

This measurement and control process continues throughout the procedure. The system periodically measures impedance through leakage current while the switching circuitry remains open. These ongoing measurements enable the system to detect if the probe moves out of the target tissue during treatment.

The combination of mechanical relay and solid-state switching elements enables both RF power control and position detection through the same circuit paths. The mechanical relay provides electrical isolation when needed, while the SSR characteristics enable impedance measurement without requiring a closed circuit. The use of leakage current through open solid-state relays provides distinct measurement capabilities. While a typical solid-state relay in an open state would be expected to block current flow, the inherent characteristics of these components enable a small current to pass through. The system leverages this behavior for probe position detection.

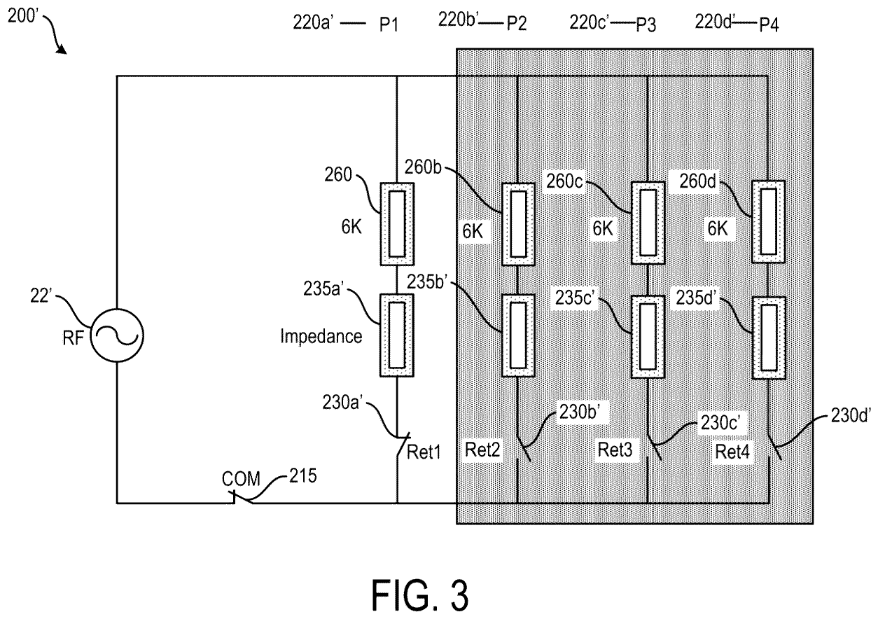

FIG. 3 shows an exemplary schematic electrical diagram 200′ illustrating the equivalent impedance values of solid-state relays when measuring inactive port impedance through leakage current. The diagram is similar to FIG. 2, using like elements denoted with a prime notation-RF source 22′, COM 215′, ports 220a′-220d′, source SSRs 225a′-225d′, and return SSRs 230a′-230d′. Additional impedance elements 260a-260d are shown representing the characteristic 6K ohm impedance of each SSR. The diagram demonstrates how the system utilizes the inherent leakage characteristics of solid-state relays for probe position detection.

In this configuration, each SSR exhibits an equivalent impedance of 6K ohms. The diagram represents this through 6K ohm impedance elements shown for each source SSR 225a′-225d′ (S1-S4) and return SSR 230a′-230d′ (Ret1-Ret4) pair, creating defined paths for leakage current measurement.

Between the RF source and COM point, each port contains an impedance element 235a′-235d′ that establishes a reference measurement path. This impedance element works in conjunction with the 6K ohm SSR impedances to create a measurement circuit.

When measuring through an inactive port, both the source and return SSRs maintain their open state. The leakage current flows from the RF source, through the 6K ohm impedance of the open source SSR, through the port's impedance element, and returns through the 6K ohm impedance of the open return SSR to COM.

For a probe positioned within body tissue, the total impedance measured through this leakage current path includes the combined effect of the SSR impedances, the reference impedance element, and the tissue impedance between probe electrodes. This creates characteristic measurement ranges that indicate probe position. The parallel arrangement of the measurement paths enables the system to assess each port independently. While measuring through one port, the SSR pairs for other ports remain in their open state, maintaining electrical isolation between channels.

The 6K ohm impedance value of each open SSR creates a divider with the measurement path impedance. When voltage is applied, it divides between the source SSR, return SSR, and any tissue present in the measurement path. Due to each SSR having a known impedance of 6k ohms, any additional impedance from tissue presence will change how this voltage divides across the circuit. This enables the system to detect the additional impedance presented by tissue contact, producing the measurement ranges similarly as shown in Table 1.

When applying voltages of 2.5V and 20V, the leakage current flowing through these defined impedance paths generates measurable voltage drops. These voltage drops, when measured across the known SSR impedances, indicate whether a probe contacts body tissue. Through the combination of SSR equivalent impedances and reference impedance elements, the system establishes known current paths for position detection without requiring closed switching circuits. This enables continuous monitoring of probe position while maintaining the capability for RF power delivery.

The impedance relationship between open SSRs and tissue contact creates measurement patterns. When a probe remains outside body tissue, the measured impedance primarily reflects the series combination of SSR impedances. However, when a probe contacts tissue, the parallel tissue impedance path alters the total measured impedance.

In some embodiments, the system can utilize different voltage levels for position detection while maintaining the same SSR configuration. While 2.5V and 20V represent example test voltages, other voltage combinations may be selected based on the specific measurement requirements.

In some embodiments, the system may employ variable measurement timing based on procedural phase. More frequent measurements through the leakage current paths may occur during initial probe positioning, while periodic checks during stable therapy delivery.

In some embodiments, the system can implement adaptive impedance thresholds. While fixed ranges indicate basic in-body versus out-of-body position, the system may adjust these thresholds based on measured tissue characteristics or procedural requirements.

The measurement configuration shown in FIG. 3 demonstrates how solid-state relay characteristics enable probe position detection through leakage current paths. By maintaining known impedance relationships in the open state, the system provides position feedback without requiring closed measurement circuits.

This approach to position detection through leakage current measurement can monitor probe position without interrupting therapy capability, maintain electrical isolation between channels, and provide continuous position feedback throughout procedures. The configuration of impedance elements and solid-state relays creates measurement paths while preserving system safety features.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

1. A tissue ablation system, comprising:
a radiofrequency generator having a power source and a controller operatively coupled to the power source and having a processor and a memory element;
the radiofrequency generator having a plurality of connector ports, wherein each of the plurality of connector ports has a plurality of electrical pins;
a radiofrequency probe electrically coupled to a first one of the plurality of connector ports and having first and second electrodes connected to corresponding ones of the plurality of electrical pins;
wherein the power source is configured to deliver radiofrequency power to the electrodes sufficient to provide controlled heating of tissue during a surgical procedure;
wherein the processor is configured to execute program instructions to:
detect a leakage current between different electrical pins of the plurality of connector ports;
measure an impedance value between the electrodes based on the detected leakage current;
compare the measured impedance value to one or more predetermined impedance ranges; and
determine whether the radiofrequency probe is positioned within body tissue based on the comparison.

2. The tissue ablation system of claim 1, wherein the processor is further configured to:
apply a first voltage level between the electrical pins;
measure a first impedance value;
apply a second voltage level between the electrical pins;
measure a second impedance value; and
determine the probe position based on changes between the first and second impedance values.

3. The tissue ablation system of claim 2, wherein the second voltage level is higher than the first voltage level.

4. The tissue ablation system of claim 1, wherein the predetermined impedance ranges comprise:
a first range between approximately 2000 ohms and 3500 ohms indicating the probe is positioned within body tissue; and
a second range above approximately 16000 ohms indicating the probe is positioned outside body tissue.

5. The tissue ablation system of claim 1, wherein the processor is configured to:
periodically detect leakage current between the electrical pins to monitor probe position during a procedure.

6. The tissue ablation system of claim 1, wherein the processor is configured to:
detect when measured impedance values indicate the probe has been removed from body tissue; and
prevent activation of radiofrequency power delivery when the probe is detected outside body tissue.

7. The tissue ablation system of claim 1, further comprising:
a second radiofrequency probe electrically coupled to a second one of the plurality of connector ports and having third and fourth electrodes connected to corresponding ones of the plurality of electrical pins.

8. The tissue ablation system of claim 7, wherein the processor is configured to:
detect leakage current between electrical pins connected to the third and fourth electrodes;
measure impedance values based on the detected leakage current; and
determine position of the second radiofrequency probe based on the measured impedance values.

9. The tissue ablation system of claim 1, wherein measuring the impedance value comprises:
detecting drop across the pins connected to the first and second electrodes; and
calculating the impedance based on the detected voltage drop and the detected leakage current.

10. The tissue ablation system of claim 1, wherein the processor is configured to:
measure impedance values between approximately 110 ohms and 4200 ohms when the probe is positioned within body tissue.

11. The tissue ablation system of claim 2, wherein the first voltage level is approximately 2.5V and the second voltage level is approximately 20V.

12. A tissue ablation system comprising:
a radiofrequency generator comprising a power source and a controller operatively coupled to the power source and having a processor and a memory element, the radiofrequency generator having a plurality of connector ports, wherein each of the plurality of connector ports has a plurality of electrical pins;
a first radiofrequency probe electrically coupled to a first one of the plurality of connector ports to form a first channel and having first and second electrodes connected to corresponding ones of the plurality of electrical pins;
a second radiofrequency probe electrically coupled to a second one of the plurality of connector ports to form a second channel and having third and fourth electrodes connected to corresponding ones of the plurality of electrical pins;
wherein the power source is configured to deliver radiofrequency power to the electrodes via the corresponding electrical pins;
wherein the processor is configured to execute program instructions to:
detect leakage current between electrical pins of the plurality of connector ports;
measure impedance values based on the detected leakage current;
determine positions of the first and second radiofrequency probes based on the measured impedance values; and
control delivery of radiofrequency power based on the determined positions.

13. The tissue ablation system of claim 12, wherein the controller is configured to:
apply a first voltage level of approximately 2.5V across the pins of each channel;
measure first impedance values;
apply a second voltage level of approximately 20V across the pins of each channel;
measure second impedance values; and
determine probe positions based on changes between the first and second impedance values.

14. The tissue ablation system of claim 12, wherein the controller is configured to:
compare measured impedance values to stored impedance ranges corresponding to in-body and out-of-body probe positions.

15. The tissue ablation system of claim 14, wherein:
the in-body impedance range is between approximately 2000 ohms and 3500 ohms; and
the out-of-body impedance range is above approximately 16000 ohms.

16. The tissue ablation system of claim 12, wherein the controller is configured to:
periodically detect leakage current between electrical pins to monitor probe positions during a procedure.

17. A computer-implemented method of detecting probe position in a tissue ablation system having a radiofrequency generator with a plurality of connector ports, wherein each connector port has a plurality of electrical pins, and a radiofrequency probe having first and second electrodes connected to corresponding pins of a first connector port, the method comprising:
executing, by a processor of a controller operatively coupled to the radiofrequency generator, program instructions stored in a memory element to:
detect leakage current between the pins connected to the first and second electrodes;
measure impedance values based on the detected leakage current;
compare the measured impedance values to predetermined threshold values;
determine whether the probe is positioned within body tissue based on the comparison; and
control delivery of radiofrequency energy based on the determined probe position.

18. The computer-implemented method of claim 17, wherein executing the program instructions further comprises:
applying different voltage levels across the pins while detecting leakage current;
measuring impedance values at each voltage level; and
determining probe position based on changes in measured impedance across the different voltage levels.

19. The computer-implemented method of claim 17, wherein executing the program instructions further comprises:
comparing measured impedance values to:
a first range between approximately 2000 ohms and 3500 ohms indicating in-body probe position; and
a second range above approximately 16000 ohms indicating out-of-body probe position.

20. The computer-implemented method of claim 17, wherein executing the program instructions further comprises:
continuously monitoring impedance through leakage current detection during a procedure;
detecting when measured impedance indicates probe removal from body tissue; and
preventing activation of radiofrequency power delivery when the probe is detected outside body tissue.
